(19) United States
(12) Patent Application Publication
(10) Pub. No.: US 2018/0243486 A1
(43) Pub. Date: Aug. 30, 2018

(54) DRAIN SUPPORT APRON

(71) Applicant: Lea M. Blackwell, Fort Myers, FL (US)

(72) Inventor: Lea M. Blackwell, Fort Myers, FL (US)

(21) Appl. No.: 15/627,770

(22) Filed: Jun. 20, 2017

Related U.S. Application Data
(63) Continuation-in-part of application No. 15/443,732, filed on Feb. 27, 2017, now Pat. No. 9,854,851.

(51) Int. Cl. A61M 1/00

(52) U.S. Cl. CPC A61M 1/0009; A61M 2202/0405; A61M 2205/0216; A61M 2209/088; A61M 2202/0413

(57) ABSTRACT

A drain support apron (1) having a main panel (2) with a plurality of pockets (9) located thereon that provide storage for one or more drain bulbs (10). The drain apron is preferably worn around a patient's midsection.

### CROSS-REFERENCE TO RELATED APPLICATIONS

This application is a continuation of application Ser. No. 15/443,732 filed on Feb. 27, 2017, which issued as U.S. Pat. No. 9,854,851 and is a continuation of application number 14/943,560 filed on Nov. 17, 2015, which issued as U.S. Pat. No. 9,578,902 and is a continuation of application Ser. No. 14/308,215 filed on Jun. 18, 2014, which issued as U.S. Pat. No. 9,277,773 and is a continuation in part of application Ser. No. 13/289,016 filed on Nov. 4, 2011, which issued as U.S. Pat. No. 8,790,154. The patent applications identified above are incorporated herein by reference in their entirety to provide continuity of disclosure.

### FIELD OF THE INVENTION

This invention relates to surgical drains and drain bulbs used for collecting fluids and more particularly a device worn around the abdomen of the patient used for supporting drain bulbs on the patient's body to prevent pulling on drain tubes inserted into the patient.

### BACKGROUND OF THE INVENTION

Surgical drains are tubes used to remove lymphatic fluid, infected fluid, blood or other fluids in surgical wounds. Drains are used for a wide variety of surgical procedures including breast surgery, melanoma surgery, abdominoplasties, breast reductions and vascular surgery. Drains are commonly placed by surgeons to drain body fluid which accumulates in the space created by surgery, to remove infection, and/or to remove accumulated blood. Drain tubes are connected to reservoir bulbs used to collect fluids from a surgical site and incision. The drain bulbs are reservoirs connected to a flexible drainage tube sutured into place or affixed with adhesive at the site the drain exits the skin. Drain bulbs remove fluid from the surgical wound through negative pressure suction. Typically, drains are left in a surgical site for one or two weeks.

A problem commonly experienced by patient's and medical staff caring for the patients, is that the drain bulbs collecting the fluid can become heavy and pull on the site where the drain exits the skin. The length of the drainage tube causes the drains to be mobile and they can be pulled, thereby causing further injury and/or discomfort to the patient. The drain bulbs are difficult to manage in showers especially if there are multiple drains.

Therefore, a need exists for a drain support apron that holds drain bulbs securely to a patient's body to prevent the drain bulbs from falling or pulling unnecessarily on the patient's drain tubes.

### SUMMARY OF THE INVENTION

The primary object of the present invention is to provide a drain support apron that fits comfortably around a patient's abdomen and provides secure storage for placement of one or more drain bulbs on the patient's body.

The present invention fulfills the above and other objects by providing a drain support apron having a main panel with a plurality of pockets located thereon that provide storage for one or more drain bulbs. The drain apron is preferably worn around a patient's midsection. Straps extend from side edges of the main panel of the drain support apron and are wrapped around the patient's midsection and back and secured on the patient's side, so the patient does not have to lay on the buckle or other attachment mechanism securing the two straps to each other. Elastic is located along an upper edge of each pocket to prevent the drain bulbs from falling out of the apron, which would cause unwanted pulling on the sutures holding the drain tubes in the surgical wound.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a front view of a drain support apron of the present invention;

FIG. 2 is a front view of a drain support apron of the present invention being worn by a patient;

FIG. 3 is a front view of a drain support apron of the present invention being worn by a patient; and

FIG. 4 is a front view of a drain support apron of the present invention being worn by a patient.

### DESCRIPTION OF THE PREFERRED EMBODIMENTS

For purposes of describing the preferred embodiment, the terminology used in reference to the numbered accessories in the drawings is as follows:

1. drain support apron, generally

2. main panel

3. front surface of main panel

4. rear surface of main panel

5. top edge of main panel

6. bottom edge of main panel

7. first side edge of main panel

8. second side edge of main panel

9. pocket

10. drain bulb

11. top edge of pocket

12. elastomeric band

13. drain tube

14. surgical wound

15. shortened strap

16. first upper corner of main panel

17. first buckle

18. elongated strap

19. second upper corner of main panel

20. second buckle

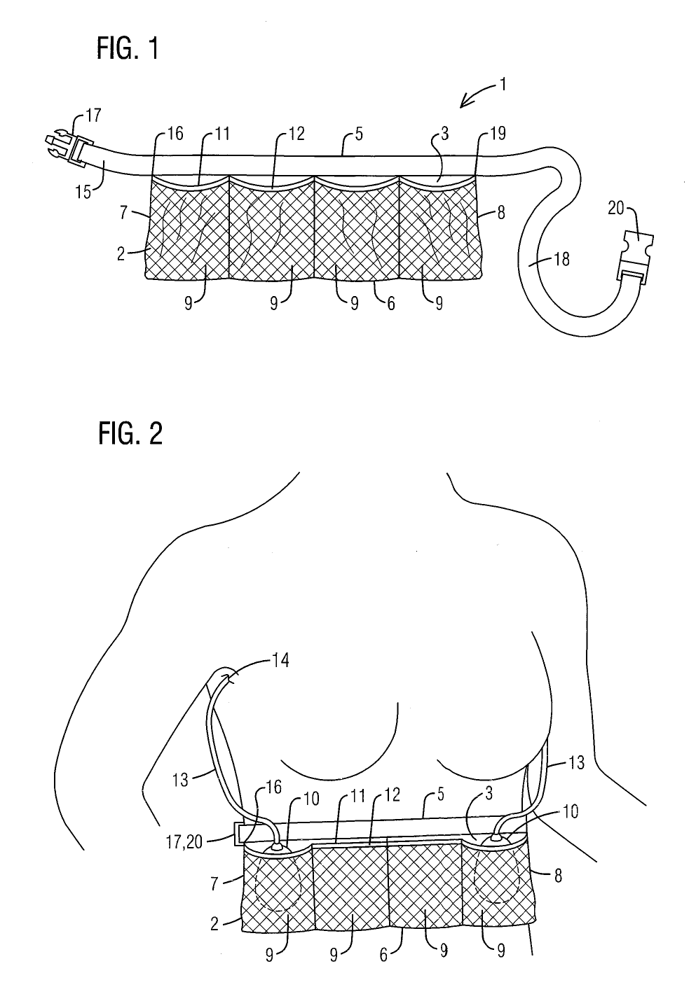

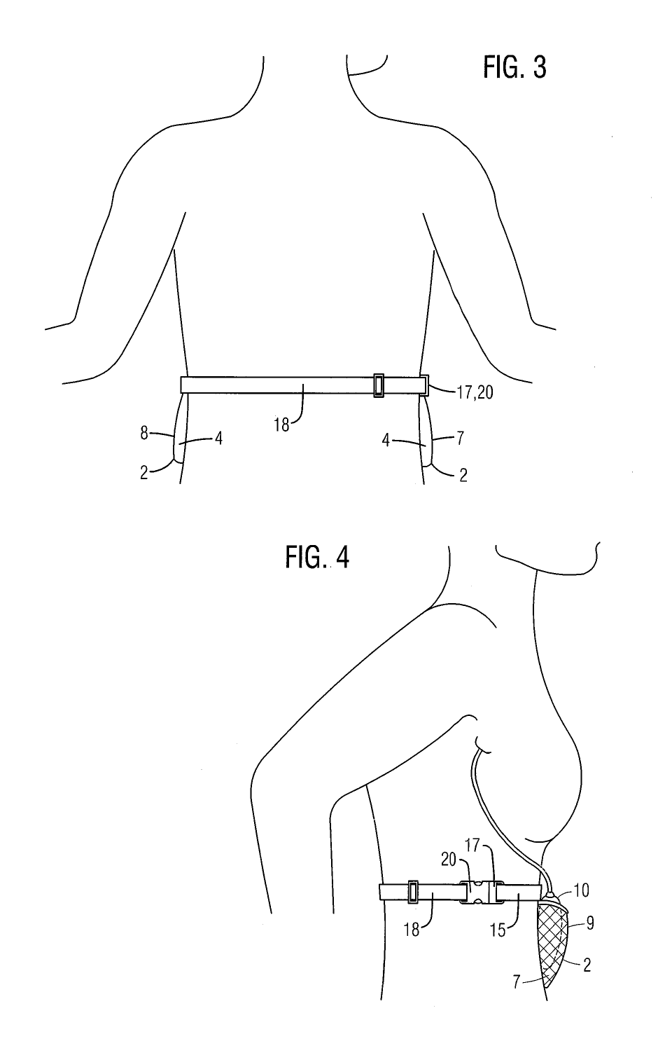

With reference to FIGS. 1-4, the drain support apron 1 comprising a substantially rectangular-shaped main panel 2 having a front surface 3, rear surface 4, top edge 5, bottom edge 6, first side edge 7 and second side edge 8. A plurality of pockets 9 are located on the front surface 3 of the main panel 2 to provide storage for one or more drain bulbs 10. Said pockets 9 are preferably rectangular-shaped and located across the front surface 3 in a row extending upward from the bottom edge 6 of the main panel 2 a predetermined distance and terminating below the top edge 5 of the main panel 2, thereby leaving a portion of the front surface 3 of the main panel 2 located between the top edge 5 of the main panel 2 and a top edge 11 of the plurality of pockets 9 exposed. One or more elastomeric bands 12 are preferably located along the top edge 11 of each pocket 9 to prevent drain bulbs 10 from falling out of the drain support apron 1, which would cause unwanted pulling on any sutures holding drain tubes 13 in a surgical wound 14.

A shortened strap 15 extends from a first upper corner 16 of the first side edge 7 of the main panel 2 and has a first buckle 17 (such as a male or female end of a buckle) or equivalent attachment means located thereon. An elongated strap 18 extends from a second upper corner 19 of the second side edge 8 of the main panel 2 and has a second buckle 20 (such as a male or female end of a buckle) or equivalent attachment means located thereon. The first buckle 17 located on the shortened strap 15 and the second buckle 20 located on the elongated strap 18 are used to engage each other to secure the drain support apron 1 around a patient's midsection. The shortened strap 15 has a length that is shorter than the elongated strap 18. The shortened strap 15 in combination with the elongated strap 18 causes the first buckle 17 and second buckle 20 to be positioned on the patient's side so the patient does not have to lay on the first buckle 17 and second buckle 20 or equivalent attachment means securing the shortened strap 15 to the elongated strap 18. Alternatively, the first buckle 17 may be attached directly to the first side edge 7 of the main panel 2 or adjacent thereto via strapping or other attachment means necessary for attaching the first buckle 17 to the main panel 2.

The main panel 2 is preferably constructed out of a non-stretchable water resistant material and/or quick drying material, such as nylon. The plurality of pockets 9, are preferably constructed out of a netting or mesh material having elastomeric qualities that allow the pockets 9 to stretch over any drain bulbs 10 inserted inside. The mesh material also allows the drain bulbs 10 and contents thereof to be visually inspected without having to remove the drain bulbs 10 from the pockets 9.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

1. A drain support apron for surgical drain bulbs comprising:
a substantially rectangular-shaped main panel having a front surface, a rear surface, top edge, bottom edge, first side edge and second side edge;
a row of a plurality of pockets located across the front surface of the main panel to provide storage for one or more surgical drain bulbs;
said plurality of pockets being immediately adjacent to each other wherein a vertical side edge of each pocket shares a vertical side edge of an adjacent pocket;
a shortened strap extending from a first upper corner of the first side edge of the main panel;
a first buckle located on the shortened strap;
an elongated strap extending from a second upper corner of the second side edge of the main panel;
a second buckle located on the elongated strap; and
said shortened strap having a length that is shorter than the elongated strap.

2. The drain support apron of claim 1 wherein:
said pockets located across the front surface of the main panel extend upward from the bottom edge of the main panel a predetermined distance and terminate below the top edge of the main panel, thereby leaving a portion of the front surface of the main panel located between the top edge of the main panel and a top edge of the plurality of pockets exposed.

3. The drain support apron of claim 1 further comprising:
one or more elastomeric bands located along the top edge of each pocket to prevent surgical drain bulbs from falling out of the drain support apron.

4. The drain support apron of claim 1 wherein:
said main panel is constructed out of a non-stretchable material; and
said plurality of pockets are constructed out of a mesh material having elastomeric qualities.

5. A drain support apron for surgical drain bulbs comprising:
a substantially rectangular-shaped main panel having a front surface, a rear surface, top edge, bottom edge, first side edge and second side edge;
a row of a plurality of pockets located across the front surface of the main panel to provide storage for one or more surgical drain bulbs;
a shortened strap extending from a first upper corner of the first side edge of the main panel;
a first buckle located on the shortened strap;
an elongated strap extending from a second upper corner of the second side edge of the main panel;
a second buckle located on the elongated strap;
said shortened strap having a length that is shorter than the elongated strap;
said pockets located across the front surface of the main panel extend upward from the bottom edge of the main panel a predetermined distance and terminate below the top edge of the main panel, thereby leaving a portion of the front surface of the main panel located between the top edge of the main panel and a top edge of the plurality of pockets exposed;
said plurality of pockets being immediately adjacent to each other wherein a vertical side edge of each pocket shares a vertical side edge of an adjacent pocket; and
an elastomeric band located along the top edge of each pocket to prevent drain bulbs from falling out of the drain support apron.

6. (canceled)

7. The drain support apron of claim 5 wherein:
said main panel is constructed out of a non-stretchable material; and
said plurality of pockets are constructed out of a mesh material having elastomeric qualities.

8. (canceled)

9. (canceled)

10. (canceled)

11. (canceled)

12. A drain support apron for surgical drain bulbs comprising:
a substantially rectangular-shaped main panel having a front surface, a rear surface, top edge, bottom edge, first side edge and second side edge;
a row of a plurality of pockets located across the front surface of the main panel to provide storage for one or more surgical drain bulbs;
a first strap extending from a first upper corner of the first side edge of the main panel;
a first buckle located on the first strap;
a second strap extending from a second upper corner of the second side edge of the main panel;
a second buckle located on the first strap;
said pockets located across the front surface of the main panel extend upward from the bottom edge of the main panel a predetermined distance and terminate below the top edge of the main panel, thereby leaving a portion of the front surface of the main panel located between the top edge of the main panel and a top edge of the plurality of pockets exposed;
said plurality of pockets being immediately adjacent to each other wherein a vertical side edge of each pocket shares a vertical side edge of an adjacent pocket;
an elastomeric band located along the top edge of each pocket to prevent drain bulbs from falling out of the drain support apron; and
said main panel is constructed out of a non-stretchable material; and
said plurality of pockets are all constructed out of a mesh material having elastomeric qualities.
